(19) United States
(12) Plant Patent Application Publication
(10) Pub. No.: US 2006/0010547 P1
(43) Pub. Date: Jan. 12, 2006

(54) OSTEOSPERMUM PLANT NAMED 'OSTE LIGHTPUR'

(50) OSTEOSPERMUM ECKLONIS; OSTEOSPERMUM

(75) Inventor: Pim Kaagman, PE Andijk (NL)

Correspondence Address: JONDLE & ASSOCIATES P.C., 858 HAPPY CANYON ROAD SUITE 230, CASTLE ROCK, CO, 80108, US

(73) Assignee: Goldsmith Seeds, Inc.

(21) Appl. No.: 10/885,565

(22) Filed: Jul. 6, 2004

(51) Int. Cl. A01H 5/00

(52) U.S. Cl. PLT/360

(57) ABSTRACT

An Osteospermum cultivar particularly distinguished by violet colored flowers, dark green foliage and vigorous growth is described.

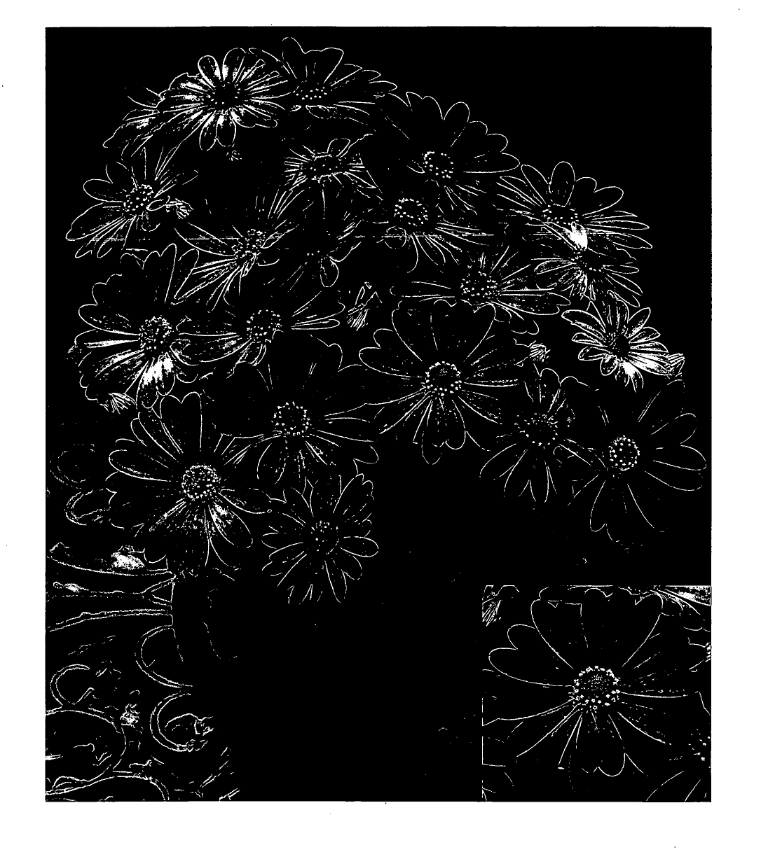

### GENUS AND SPECIES

Osteospermum ecklonis

### VARIETY DENOMINATION

‘Oste Litpur’.

### BACKGROUND OF THE NEW PLANT

The variety ‘Oste Litpur’ originated from a cross made in October, 2000. The female parent is a violet proprietary line OX-49 (unpatented). The male parent is ‘Denebola’, (patent status unknown) which is a violet commercial variety. Seed from this cross was sown in June 2001. In October 2001, single-plant selections were made for vegetative propagation using plant habit and flower color as selection criteria. One selection became the new variety ‘Oste Litpur’.

‘Oste Litpur’ is a product of a planned breeding program intended to create new Osteospermum cultivars with violet colored flowers, dark green foliage, and vigorous growth.

The new cultivar was created in 2000 in Gilroy, Calif. and has been asexually reproduced repeatedly by vegetative cuttings and tissue culture in Gilroy, Calif.; and Andijk, The Netherlands over a two ½ year period. The plant has also been trialed at Gilroy, Calif.; and Andijk, The Netherlands. The present invention has been found to retain its distinctive characteristics through successive propagation, and this novelty is firmly fixed.

### DESCRIPTION OF PHOTOGRAPH

This new Osteospermum plant is illustrated by the accompanying photograph which shows blooms, buds, and foliage of the plant in full color, the colors shown being as true as can be reasonably obtained by conventional photographic procedures. Photos were taken from plants grown in The Netherlands in the late winter/spring in a greenhouse. Plants are 4½ months old, had one terminal pinch at 3 weeks old, and are grown in a 4 inch pot with 3 treatments of cyocel.

The photograph shows the mature plant with an inset photo of a close-up of the inflorescence.

### DESCRIPTION OF THE NEW CULTIVAR

The following detailed descriptions set forth the distinctive characteristics of ‘Oste Litpur’. The data, which defines these characteristics, were collected from asexual reproductions carried out in Gilroy, Calif. The plant history was taken on 6 month old plants grown in one gallon pots in the late winter/spring season, in a LEXAN covered greenhouse under natural light and color readings were taken in the greenhouse under natural lights. Color references are primarily to the RHS Colour Chart of The Royal Horticultural Society of London (RHS) 2001. Texture description details were observed under a dissecting microscope according to The New Royal Horticultural Society Dictionary of Gardening (1992).

### THE PLANT

Classification: Osteospermum ecklonis.
Botanical: Osteospermum.
Commercial: ‘Oste Litpur’.
Form: Upright, semi-compact and mounded.
Growth and branching habit: Good vigorous, freely branched habit.
Height: From soil level to top of blooms: Approximately 37-43 cm.
Width: Approximately 25-35 cm.
Time to produce a finished flowering plant: 8-11 weeks for a 6 inch pot.
Outdoor plant performance: Herbaceous, frost tender perennial patio planters, mixed container plantings or garden beds.
Time to initiate and develop roots: Approximately 8-14 days in the greenhouse.
Root description: Fibrous, well-branched, white.

### THE LEAVES

Leaf arrangement: Alternate.
Fully expanded, Length: 5.3-5.5 cm; Width: 3.1-3.5 cm.
Leaf shape: Obovate.
Leaf margin: Dentate.
Apex aspect: Broadly acute.
Base aspect: Acuminate.
Leaf color: Upper surface — RHS 137A (green) but a littler darker; Under surface RHS 137B (green).
Leaf texture: Pilose.
Venation: Pinnate.
Venation color: RHS 144C (yellow-green) but a little lighter.
Petiole length: 0.5-0.7 cm.
Petiole diameter: 0.3 cm.
Petiole color: RHS 144C (yellow-green).
Petiole texture: Pilose.

### THE STEM

Length: 30-40 cm.
Diameter: 0.3 cm.
Stem internode length: 1.5-2.5 cm.
Color: RHS 144B (yellow-green).
Texture: Pilose.
Stem anthocyanin: None.
Peduncle color: RHS 144A (yellow-green).
Peduncle length: 7.5-14.0 cm.
Peduncle diameter: 0.2 cm.
Peduncle texture: Pilose.
Peduncle strength: Moderate to strong.

### THE BUD

Shape: Ovate.
Diameter: 0.8-0.9 cm.
Length: 1.3-1.7 cm.
Color at tight bud (starting to show color): RHS N77B (purple).

### THE FLOWER

Blooming habit: Plants flower freely from the spring through fall given the proper growing conditions.
Inflorescence/floret type: Terminal and axillary inflorescences held above the foliage. Capitulum inflorescence, radially symmetrical.
Flower diameter: 5.5-6.0 cm.
Average disc diameter: 0.1 cm.
Ray floret, immature and mature color: Upper surface is a shading mixture of RHS N74C (red-purple) and 75A (purple). Under surface is RHS N77C (purple) with longitudinal stripes of faded RHS N77A (purple).
   Floret length.—2.7-3.0 cm.
   Floret width.—0.7-0.8 cm.
   Floret apex shape.—Emarginate to Retuse.
   Floret base shape.—Cuneate.
   Floret margin.—Entire.
   Floret texture.—Papillose and hirsute on the basal margins.
   Floret quantity.—Approximately 20.
Disc floret, tube color.—RHS N155B (white) but whiter
   Shape.—Tubular, elongated.
   Length.—0.6-0.7 cm.
   Diameter at apex.—0.2 cm.
   Apex shape.—5-pointed, acute.
   Apex color.—RHS N79A (purple).
   Floret quantity.—Approximately 75.
Phyllaries, color: RHS 138A (green).
   Length.—1.0-1.2 cm.
   Width.—0.15-0.2 cm.
   Shape.—Linear.
   Apex.—Acute.
   Texture.—Pilose and glandular hairs.
   Longevity of individual blooms.—7-14 days.
   Fragrance.—None.

### THE REPRODUCTIVE ORGANS

Androecium, pollen color: RHS 13A (yellow).
   Filament color.—RHS N77C (purple).
   Length of stamen.—0.3-0.5 cm.
   Location of androecium.—Disc floret only.
Gynoecium, pistil: 1 per floret.
   Pistil length.—0.4 cm.
   Stigma color.—RHS N79A (purple).
   Style color.—RHS N155D (white) but whiter.
   Location of gynoecium.—Both disc and ray florets.
   Fruit seed set.—Has not been observed.

### DISEASE AND INSECT RESISTANCE

Has not been observed.

### COMPARISON WITH KNOWN CULTIVARS

‘Oste Litpur’ differs from the female parent, OX-49 in the following ways: ‘Oste Litpur’ is later to flower, more vigorous and a lighter flower color than OX-49.

‘Oste Litpur’ differs from the male parent, ‘Denebola’ in the following ways: ‘Oste Litpur’ is earlier to flower, has better branching and shorter flower stems than ‘Denebola’.
‘Oste Litpur’ differs from the commercial Osteospermum variety ‘Sunny Martha’ in the following ways: ‘Oste Litpur’ is more compact and earlier to flower than ‘Sunny Martha.’ In addition, ‘Oste Litpur’ has smaller leaves, lighter foliage, more branches and more flowers than ‘Sunny Martha.’

I claim:

1. A new and distinct cultivar of Osteospermum plant as shown and described herein.
